(19) United States
(12) Patent Application Publication
(10) Pub. No.: US 2025/0382243 A1
(43) Pub. Date: Dec. 18, 2025

(54) PREPARATION AND STORAGE OF ETHYLENE OLIGOMERIZATION CATALYST COMPOSITIONS

(71) Applicant: Chevron Phillips Chemical Company LP, The Woodlands, TX (US)

(72) Inventors: Michael S. Webster-Gardiner, Kingwood, TX (US); Steven M. Bischof, Kingwood, TX (US)

(21) Appl. No.: 19/234,405

(22) Filed: Jun. 11, 2025

Related U.S. Application Data
(60) Provisional application No. 63/660,151, filed on Jun. 14, 2024.

(51) Int. Cl. C07C 2/36; B01J 31/14; B01J 31/18; B01J 31/22; B01J 37/04

(52) U.S. Cl. CPC C07C 2/36; B01J 31/143; B01J 31/189; B01J 31/2226; B01J 37/04; B01J 2231/20; B01J 2531/004; B01J 2531/62; C07C 2531/24

(57) ABSTRACT

Preparations for storage-stable activated catalyst compositions comprising chromium complexes are disclosed herein. Oligomerization processes using the activated catalyst compositions produce a reduced amount of undesirable byproducts such as fouling solids, without a concomitant reduction in catalyst activity, yield, or selectivity.

### REFERENCE TO RELATED APPLICATION

This application claims the benefit of U.S. Provisional Patent Application No. 63/660,151, filed on Jun. 14, 2024, the disclosure of which is incorporated herein by reference in its entirety.

### TECHNICAL FIELD

The present disclosure relates generally to processes for preparing activated catalyst compositions, and the use of the catalyst compositions in ethylene oligomerization processes.

### BACKGROUND

Alpha olefins such as 1-hexene and 1-octene can be produced using an ethylene reactant and various combinations of catalyst systems and oligomerization processes. It can be beneficial for the catalyst system to be storage stable in an activated form, thereby allowing increased control over oligomerization processes. Accordingly, it is to these ends that the present invention is generally directed.

### SUMMARY

This summary is provided to introduce a selection of concepts in a simplified form that are further described herein. This summary is not intended to identify required or essential features of the claimed subject matter. Nor is this summary intended to be used to limit the scope of the claimed subject matter.

Disclosed herein are processes for preparing an activated catalyst composition, the process comprising (a) contacting a mixture of a chromium complex in an aromatic hydrocarbon solvent with an aluminoxane at an activation temperature to form the activated catalyst composition; and (b) optionally, reducing a temperature of the activated catalyst composition to a storage temperature. In certain aspects, at least one of the activation temperature and the storage temperature is less than 20° C. (e.g., less than 0° C.; or in a range from −80° C. to 20° C., or in a range from −40° C. to 0° C.). As a result of the preparation according to this disclosure, the activated catalyst composition can be stable at the storage temperature for greater than 24 hours (e.g., from 2 to 30 days) without loss of activity.

The activated catalyst compositions prepared as disclosed herein also may be employed within oligomerization processes. In certain aspects, the oligomerization processes can comprise (i) contacting ethylene, an activated catalyst composition, an organic reaction medium, and optionally hydrogen, in an oligomerization reactor under oligomerization conditions, (ii) forming an oligomer product in the oligomerization reactor, and (iii) discharging an effluent stream from the oligomerization reactor, the effluent stream comprising unreacted ethylene and the oligomer product. In certain aspects, oligomerization processes conducted according to the present disclosure can result in a reduced amount of fouling solids, increased catalytic activity, increased oligomer productivity, or any combination thereof.

Both the foregoing summary and the following detailed description provide examples and are explanatory only. Accordingly, the foregoing summary and the following detailed description should not be considered to be restrictive. Further, features or variations may be provided in addition to those set forth herein. For example, certain aspects may be directed to various feature combinations and sub-combinations described in the detailed description.

### BRIEF DESCRIPTION OF THE FIGURES

The following figures form part of the present specification and is included to further demonstrate certain aspects of the present invention. The invention may be better understood by reference to these figures in combination with the detailed description.

FIG. 1 presents a chart showing the results of Examples 1-6, with respect to the amount of fouling solids produced at different activation temperatures.

FIG. 2 presents a chart showing the results of Examples 7-11, with respect to the oligomer productivity of ethylene oligomerization processes conducted using various storage times for the activated catalyst composition.

FIGS. 3-4 present charts showing the results of Examples 12-13, with respect to the amount of fouling solids at different activation temperatures.

While the inventions disclosed herein are susceptible to various modifications and alternative forms, only a few specific aspects have been shown by way of example in the drawing and described in detail below. The figures and detailed description of specific aspects are not intended to limit the breadth or scope of the inventive concepts or the appended claims in any manner. Rather, the figures and detailed description are provided to illustrate the inventive concepts to a person of ordinary skill in the art and to enable such person to make and use the inventive concepts.

### DEFINITIONS

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2nd Ed (1997), can be applied, as long as that definition does not conflict with any other disclosure or definition applied herein, or render indefinite or non-enabled any claim to which that definition is applied. To the extent that any definition or usage provided by any document incorporated herein by reference conflicts with the definition or usage provided herein, the definition or usage provided herein controls.

Herein, features of the subject matter are described such that, within particular aspects, a combination of different features can be envisioned. For each and every aspect and each and every feature disclosed herein, all combinations that do not detrimentally affect the compounds, compositions, processes, or methods described herein are contemplated with or without explicit description of the particular combination. Additionally, unless explicitly recited otherwise, any aspect or feature disclosed herein can be combined to describe inventive compounds, compositions, processes, or methods consistent with the present disclosure.

In this disclosure, while compositions and processes/methods are described in terms of “comprising” various materials or components and steps, the compositions and processes/methods also can “consist essentially of” or “consist of” the various materials or components and steps, unless stated otherwise. For example, a catalyst composition consistent with aspects of the present invention can comprise; alternatively, can consist essentially of; or alternatively, can consist of; a chromium complex, an aromatic hydrocarbon solvent, and an aluminoxane compound. The terms “a,” “an,” and “the” are intended to include plural alternatives, e.g., at least one, unless otherwise specified. For instance, the disclosure of “an aluminoxane compound” is meant to encompass one, or mixtures or combinations of two or more, aluminoxane compound(s), unless otherwise specified.

For any generic or specific compound or group disclosed herein, any name or structure presented is intended to encompass all conformational isomers, regioisomers, stereoisomers, and mixtures thereof that can arise from a particular set of substituents, unless otherwise specified. The name or structure also encompasses all enantiomers, diastereomers, and other optical isomers (if there are any), whether in enantiomeric or racemic forms, as well as mixtures of stereoisomers, as would be recognized by a skilled artisan, unless otherwise specified. For example, a general reference to hexene (or hexenes) includes all linear or branched, acyclic or cyclic, hydrocarbon compounds having six carbon atoms and 1 carbon-carbon double bond; a general reference to pentane includes n-pentane, 2-methyl-butane, and 2,2-dimethylpropane; and a general reference to a butyl group includes an n-butyl group, a sec-butyl group, an iso-butyl group, and a t-butyl group.

The terms “contacting” and “combining” are used herein to describe compositions and processes/methods in which the materials are contacted or combined together in any order, in any manner, and for any length of time, unless otherwise specified. For example, the materials can be blended, mixed, slurried, dissolved, reacted, treated, impregnated, compounded, or otherwise contacted or combined in some other manner or by any suitable method or technique.

The term “hydrocarbon” whenever used in this specification and claims refers to a compound containing only carbon and hydrogen, whether saturated or unsaturated. Other identifiers can be utilized to indicate the presence of particular groups in the hydrocarbon (e.g., halogenated hydrocarbon indicates that the presence of one or more halogen atoms replacing an equivalent number of hydrogen atoms in the hydrocarbon). The term “hydrocarbyl group” is used herein in accordance with the definition specified by IUPAC: a univalent group formed by removing a hydrogen atom from a hydrocarbon (that is, a group containing only carbon and hydrogen). Non-limiting examples of hydrocarbyl groups include alkyl, alkenyl, aryl, and aralkyl groups, amongst other groups.

The term “oligomer” refers to a compound that contains from 2 to 20 monomer units. The terms “oligomerization product” and “oligomer product” include all products made by the “oligomerization” process, including the “oligomers” and products which are not “oligomers” (e.g., products which contain more than 20 monomer units, or solid polymer), but exclude other non-oligomer components of an oligomerization reactor effluent stream, such as unreacted ethylene, organic reaction medium, and hydrogen, amongst other components.

The terms “catalyst composition,” “catalyst mixture,” “catalyst system,” and the like, do not depend upon the actual product or composition resulting from the contact or reaction of the initial components of the disclosed or claimed catalyst composition (or catalyst mixture or catalyst system), the nature of the activated catalyst composition, or the fate of the aromatic hydrocarbon solvent, and the chromium complex after combining these components. Therefore, the terms “catalyst composition,” “catalyst mixture,” “catalyst system,” and the like, encompass the initial starting components of the composition, as well as whatever product(s) may result from contacting these initial starting components, and this is inclusive of both heterogeneous and homogenous catalyst systems or compositions. The terms “catalyst composition,” “catalyst mixture,” “catalyst system,” and the like, may be used interchangeably throughout this disclosure.

Several types of ranges are disclosed in the present invention. When a range of any type is disclosed or claimed, the intent is to disclose or claim individually each possible number that such a range could reasonably encompass, including end points of the range as well as any sub-ranges and combinations of sub-ranges encompassed therein. For example, the molar ratio of Al:Cr in the activated catalyst composition can be in various ranges. By a disclosure that the molar ratio can be in a range from 10:1 to 5,000:1, the intent is to recite that the molar ratio can be any molar ratio within the range and, for example, can include any range or combination of ranges from 10:1 to 5,000:1, such as from 50:1 to 3,000:1, from 75:1 to 2,000:1, from 100:1 to 2,000:1, or from 100:1 to 1,000:1, and so forth. Likewise, all other ranges disclosed herein should be interpreted in a manner similar to this example.

In general, an amount, size, formulation, parameter, range, or other quantity or characteristic is “about” or “approximate” whether or not expressly stated to be such. Whether or not modified by the term “about” or “approximately,” the claims include equivalents to the quantities or characteristics.

Although any methods and materials similar or equivalent to those described herein can be used in the practice or testing of the invention, the typical methods and materials are herein described.

All publications and patents mentioned herein are incorporated herein by reference in their entirety for the purpose of describing and disclosing, for example, the constructs and methodologies that are described in the publications and patents, which might be used in connection with the presently described invention.

### DETAILED DESCRIPTION

Disclosed herein are activated catalyst compositions containing chromium complexes and ethylene oligomerization processes utilizing the activated catalyst compositions to produce 1-hexene and/or 1-octene.

### Processes for Preparing Activated Catalyst Compositions

Processes for preparing an activated catalyst composition consistent with aspects of this invention can comprise (a) contacting a mixture of a chromium complex in an aromatic hydrocarbon solvent with an aluminoxane at an activation temperature to form the activated catalyst composition, and (b) optionally, reducing a temperature of the activated catalyst composition to a storage temperature. At least one of the activation temperature and the storage temperature can be less than 20° C. (e.g., less than 0° C.; or in a range from −80° C. to 20° C., or in a range from −40° C. to 0° C.). The activated catalyst composition can be stable at the storage temperature for greater than 24 hours (e.g., from 2 to 30 days) without loss of activity.

The activation temperature was found to have the surprising effect of suppressing the amount of fouling solids generated during the oligomerization reactions, without negatively impacting the productivity, selectivity, and purity of the desired oligomer products. Generally, lowering the activation temperature to less than 20° C., less than 0° C., less than −10° C., less than −20° C., less than −30° C., less than −40° C., less than −55° C., or less than −75° C., led to the surprising reduction of fouling solids. Alternatively, the activation temperature can be in a range from −80° C. to 20° C., from −80° C. to 10° C., from −60° C. to 0° C., from −40° C. to 0° C., or from −20° C. to 0° C.

Alternatively, the activation temperature may be 20° C., or greater than 20° C. Such processes can further comprise reducing the temperature activated catalyst composition to a storage temperature. In certain aspects, the storage temperature generally can be any as described above for the activation temperature, and including storage temperatures less than 20° C., less than 0° C., less than −10° C., less than −20° C., less than −30° C., less than −40° C., less than −55° C., or less than −75° C., which also led to the surprising reduction of fouling solids in subsequent oligomerization processes. In other aspects, the storage temperature can be in a range from −80° C. to 20° C., from −80° C. to 10° C., from −60° C. to 0° C., from −40° C. to 0° C., or from −20° C. to 0° C.

Processes disclosed herein may also lead to an increased storage stability of the activated catalyst composition. In certain aspects, the activated catalyst composition may be stored prior to its use in oligomerization processes such as those described below. In certain aspects, activated catalyst compositions may have an activity, oligomer productivity, yield, and/or purity that is within 10% of or greater than that of a freshly activated catalyst composition prepared at room temperature. In certain aspects, processes disclosed herein can comprise a storage time for the activated catalyst of greater than or equal to 8 hours, 12 hours, 24 hours, 2 days, 3 days, 5 days, or 7 days.

In certain aspects, the aromatic hydrocarbon solvent can be the same or different from the organic reaction medium described below. The aromatic hydrocarbon solvent can comprise benzene, toluene, cumene, ethylbenzene, xylene (m-xylene, o-xylene, p-xylene, or mixtures thereof), styrene, mesitylene, and the like. Combinations of two or more aromatic hydrocarbons can be utilized, if desired.

In an aspect, the aluminoxane utilized in the catalyst systems can comprise, can consist essentially of, or can consist of, any aluminoxane which in conjunction with the heteroatomic ligand chromium complex can catalyze the formation of an oligomer product. In a non-limiting aspect, the aluminoxane can have a repeating unit characterized by Formula (III):

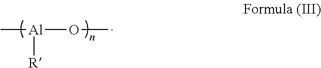

In formula (III), R′ is a linear or branched alkyl group. Alkyl groups of aluminoxanes (and alkylaluminum compounds) are independently described herein and can be utilized without limitation to further describe the aluminoxanes having Formula (III) and/or the alkylaluminum compounds. Generally, n of Formula (III) can be greater than 1; or alternatively, greater than 2. In an aspect, n can range from 2 to 15; or alternatively, range from 3 to 10.

In a non-limiting aspect, the aluminoxane can comprise, consist essentially of, or consist of, methylaluminoxane (MAO), ethylaluminoxane, modified methylaluminoxane (MMAO), n-propylaluminoxane, iso-propyl-aluminoxane, n-butylaluminoxane, sec-butylaluminoxane, iso-butylaluminoxane, t-butylaluminoxane, 1-pentylaluminoxane, 2-pentylaluminoxane, 3-pentyl-aluminoxane, iso-pentyl-aluminoxane, neopentylaluminoxane, or mixtures thereof. In some non-limiting aspects, the aluminoxane can comprise, consist essentially of, or consist of, methylaluminoxane (MAO), modified methylaluminoxane (MMAO), isobutyl aluminoxane, t-butyl aluminoxane, or mixtures thereof. In other non-limiting aspects, the aluminoxane can comprise, consist essentially of, or consist of, methylaluminoxane (MAO); alternatively, ethylaluminoxane; alternatively, modified methylaluminoxane (MMAO); alternatively, n-propylaluminoxane; alternatively, iso-propyl-aluminoxane; alternatively, n-butylaluminoxane; alternatively, sec-butylaluminoxane; alternatively, iso-butylaluminoxane; alternatively, t-butyl aluminoxane; alternatively, 1-pentyl-aluminoxane; alternatively, 2-pentylaluminoxane; alternatively, 3-pentyl-aluminoxane; alternatively, iso-pentyl-aluminoxane; or alternatively, neopentylaluminoxane.

In an aspect, each alkyl group of an aluminoxane (and/or alkylaluminum compound) independently can be a C₁ to C₂₀ alkyl group; alternatively, a C₁ to C₁₀ alkyl group; or alternatively, a C₁ to C₆ alkyl group. In an aspect, each alkyl group of an aluminoxane and/or alkylaluminum compound independently can be a methyl group, an ethyl group, a propyl group, a butyl group, a pentyl group, a hexyl group, a heptyl group, or an octyl group; alternatively, a methyl group, an ethyl group, a butyl group, a hexyl group, or an octyl group. In some aspects, each alkyl group of an aluminoxane and/or alkylaluminum compound can be a methyl group, an ethyl group, an n-propyl group, an n-butyl group, an iso-butyl group, an n-hexyl group, or an n-octyl group; alternatively, a methyl group, an ethyl group, an n-butyl group, or an iso-butyl group; alternatively, a methyl group; alternatively, an ethyl group; alternatively, an n-propyl group; alternatively, an n-butyl group; alternatively, an iso-butyl group; alternatively, an n-hexyl group; or alternatively, an n-octyl group.

In certain aspects, the aluminoxane may be added to the mixture as a solution, for instance within a saturated aliphatic hydrocarbon solvent. The saturated aliphatic hydrocarbon can be a linear aliphatic hydrocarbon, a branched aliphatic hydrocarbon, or a cyclic aliphatic hydrocarbon, as well as combinations thereof. Thus, the hydrocarbon diluent and/or the organic reaction medium can comprise a linear alkane, a branched alkane, a cyclic alkane, or a combination thereof. Illustrative examples of saturated aliphatic hydrocarbons that can be utilized, either singly or in combination, include propane, butane (e.g., n-butane or isobutane), pentane (e.g., n-pentane, neopentane, cyclopentane, or isopentane), hexane, heptane, octane, cyclohexane, methyl cyclohexane, and the like, as well as combinations thereof. In a particular aspect of this disclosure, the saturated aliphatic hydrocarbon can comprise (or consist essentially of, or consist of) cyclohexane. In certain aspects, a portion of the reaction mixture may be used as the solvent described above. In another aspect of this disclosure, the saturated aliphatic hydrocarbon can comprise (or consist essentially of, or consist of) methylcyclopentane.

In other aspects, other hydrocarbon diluents may be used. Illustrative examples of linear α-olefins that can be utilized as the hydrocarbon diluent, either singly or in combination, include 1-butene, 1-hexene, 1-octene, 1-decene, 1-dodecene, 1-tetradecene, and the like, as well as combinations thereof. In a particular aspect of this disclosure, the hydrocarbon diluent can comprise a linear α-olefin that comprises (or consists essentially of, or consists of) 1-butene; alternatively, 1-hexene; alternatively, 1-octene; alternatively, 1-decene; alternatively, 1-dodecene; alternatively, 1-tetradecene; or alternatively, any mixture or combination of linear α-olefins.

Generally, where present, the alkylaluminum compound utilized in the preparation of activated catalyst compositions disclosed herein can comprise, can consist essentially of, or can consist of, a trialkylaluminum, an alkylaluminum halide, an alkylaluminum alkoxide, or any combination thereof. In some aspects, the alkylaluminum compound can comprise, can consist essentially of, or can consist of, a trialkylaluminum, an alkylaluminum halide, or any combination thereof; alternatively, a trialkylaluminum, an alkylaluminum alkoxide, or any combination thereof; or alternatively, a trialkylaluminum. In other aspects, the alkylaluminum compound can be a trialkylaluminum; alternatively, an alkylaluminum halide; or alternatively, an alkylaluminum alkoxide.

In an aspect, each alkoxide group of any alkylaluminum alkoxide disclosed herein independently can be a C₁ to C₂₀ alkoxy group, a C₁ to C₁₀ alkoxy group, or a C₁ to C₆ alkoxy group. In an aspect, each alkoxide group of any alkylaluminum alkoxide disclosed herein independently can be a methoxy group, an ethoxy group, a propoxy group, a butoxy group, a pentoxy group, a hexoxy group, a heptoxy group, or an octoxy group; alternatively, a methoxy group, an ethoxy group, a butoxy group, a hexoxy group, or an octoxy group. In some aspects, each alkoxide group of any alkylaluminum alkoxide disclosed herein independently can be a methoxy group, an ethoxy group, an n-propoxy group, an n-butoxy group, an iso-butoxy group, an n-hexoxy group, or an n-octoxy group; alternatively, a methoxy group, an ethoxy group, an n-butoxy group, or an iso-butoxy group; alternatively, a methoxy group; alternatively, an ethoxy group; alternatively, an n-propoxy group; alternatively, an n-butoxy group; alternatively, an iso-butoxy group; alternatively, an n-hexoxy group; or alternatively, an n-octoxy group.

In a non-limiting aspect, useful trialkylaluminum compounds can include trimethylaluminum, triethylaluminum, tripropylaluminum, tributylaluminum, trihexylaluminum, trioctylaluminum, or mixtures thereof. In some non-limiting aspects, useful trialkylaluminum compounds can include trimethylaluminum, triethylaluminum, tripropylaluminum, tri-n-butylaluminum, tri-isobutylaluminum, trihexylaluminum, tri-n-octylaluminum, or mixtures thereof; alternatively, triethylaluminum, tri-n-butylaluminum, tri-isobutylaluminum, trihexylaluminum, tri-n-octylaluminum, or mixtures thereof; alternatively, triethylaluminum, tri-n-butylaluminum, trihexylaluminum, tri-n-octylaluminum, or mixtures thereof. In other non-limiting aspects, useful trialkylaluminum compounds can include trimethylaluminum; alternatively, tricthylaluminum; alternatively, tripropylaluminum; alternatively, tri-n-butylaluminum; alternatively, tri-isobutylaluminum; alternatively, trihexylaluminum; or alternatively, tri-n-octylaluminum.

In a non-limiting aspect, useful alkylaluminum halides can include diethylaluminum chloride, diethylaluminum bromide, ethylaluminum dichloride, ethylaluminum sesquichloride, and mixtures thereof. In some non-limiting aspects, useful alkylaluminum halides can include diethylaluminum chloride, ethylaluminum dichloride, ethylaluminum sesquichloride, and mixtures thereof. In other non-limiting aspects, useful alkylaluminum halides can include diethylaluminum chloride; alternatively, diethylaluminum bromide; alternatively, ethylaluminum dichloride; or alternatively, ethylaluminum sesquichloride.

Referring now to the chromium complex, any suitable chromium complex can be utilized in the processes provided herein, and expect similar advantages as those identified throughout the disclosure and demonstrated by Examples 1-11 below. In certain aspects, it is contemplated that the chromium complex can comprise a heteroatomic ligand selected from a P—N—P ligand, an S—N—S ligand, and a pyrrole ligand.

In other aspects, the chromium complex can comprise an N²-phosphinyl guanidine chromium complex, an N²-phosphinyl formamidine chromium complex, an N²-phosphinyl amidine chromium complex, or any combination thereof. For instance, in certain aspects, the chromium complex can comprise, can consist essentially of, or can be, an N²-phosphinyl formamidine chromium complex, an N²-phosphinyl amidine chromium complex, an N²-phosphinyl guanidine chromium complex, a heterocyclic 2-[(phosphinyl)aminyl]imine chromium complex, or any combination thereof; alternatively, an N²-phosphinyl formamidine chromium complex; alternatively, an N²-phosphinyl amidine chromium complex; alternatively, an N²-phosphinyl guanidine chromium complex; alternatively, an N²-phosphinyl guanidine chromium complex; or alternatively, a heterocyclic 2-[(phosphinyl)aminyl]imine chromium complex.

Chromium complexes therefore can include those disclosed throughout U.S. Pat. No. 11,230,514. Further exemplary chromium complexes that may be well suited for use in the disclosed catalyst compositions and oligomerization processes include those described, for example, in U.S. Pat. Nos. 7,056,997, 7,300,904, 7,361,623, 7,554,001, 7,994,363, 8,252,956, 8,334,420, 8,471,085, 8,680,003, 8,865,610, 9,352,306, 10,464,862, 11,117,845.

In further aspects, the chromium complex can have the following formula:

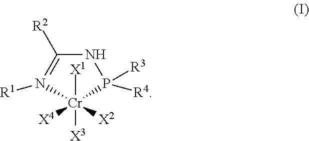

R¹, R², R³, R⁴, X¹, X², X³, and X⁴ are independently described herein and can be utilized in any combination and without limitation to further describe complexes of Formula (I).

The monoanionic ligand (X¹, X², X³, X⁴) can be a halogen (e.g., fluorine or chlorine), a carboxylate, a β-diketonate, a hydrocarboxide, a nitrate, or a chlorate. The hydrocarboxide can be an alkoxide, an aryloxide, or an aralkoxide. Generally, any carboxylate of the chromium complex independently can be a C₁ to C₂₀ carboxylate, or alternatively, a C₁ to C₁₀ carboxylate. In an aspect, each carboxylate independently can be acetate, a propionate, a butyrate, a pentanoate, a hexanoate, a heptanoate, an octanoate, a nonanoate, a decanoate, an undecanoate, or a dodecanoate; or alternatively, a pentanoate, a hexanoate, a heptanoate, an octanoate, a nonanoate, a decanoate, an undecanoate, or a dodecanoate. In some aspects, each carboxylate independently can be acetate, propionate, n-butyrate, valerate (n-pentanoate), neo-pentanoate, capronate (n-hexanoate), n-heptanoate, caprylate (n-octanoate), 2-ethylhexanoate, n-nonanoate, caprate (n-decanoate), n-undecanoate, or laurate (n-dodecanoate); alternatively, valerate (n-pentanoate), neo-pentanoate, capronate (n-hexanoate), n-heptanoate, caprylate (n-octanoate), 2-ethylhexanoate, n-nonanoate, caprate (n-decanoate), n-undecanoate, or laurate (n-dodecanoate); alternatively, capronate (n-hexanoate); alternatively, n-heptanoate; alternatively, caprylate (n-octanoate); or alternatively, 2-ethylhexanoate. In some aspects, the carboxylate can be triflate (trifluoroacetate). In other aspects, two or more of X¹, X², X³, and X⁴ can be joined to form a dianionic or trianionic or polyanionic ligand to balance the oxidation state of Cr. Where present, each β-diketonate of the chromium complex independently can be any C₁ to C₂₀ a β-diketonate; or alternatively, any C₁ to C₁₀ β-diketonate. In an aspect, each β-diketonate independently can be acetylacetonate (i.e., 2,4-pentanedionate), hexafluoroacetylacetonate (i.e., 1,1,1,5,5,5-hexafluoro-2,4-pentanedionate), or benzoylacetonate; alternatively, acetylacetonate; alternatively, hexafluoroacetylacetonate; or alternatively, benzoylacetonate.

Generally, each hydrocarboxide of the chromium complex independently can be any C₁ to C₂₀ hydrocarboxide; or alternatively, any C₁ to C₁₀ hydrocarboxide. In an aspect, each hydrocarboxide independently can be a C₁ to C₂₀ alkoxide; alternatively, a C₁ to C₁₀ alkoxide; alternatively, a C₆ to C₂₀ aryloxide; or alternatively, a C₆ to C₁₀ aryloxide. In an aspect, each alkoxide independently can be methoxide, ethoxide, a propoxide, or a butoxide; alternatively, methoxide, ethoxide, isopropoxide, or tert-butoxide; alternatively, methoxide; alternatively, an ethoxide; alternatively, an iso-propoxide; or alternatively, a tert-butoxide. In an aspect, the aryloxide can be phenoxide.

R¹, R², R³, and R⁴ are independently described herein and can be utilized in any combination and without limitation to further describe the chromium complex.

Generally, R¹ can be an organyl group: alternatively, an organyl group consisting of inert functional groups; or alternatively, a hydrocarbyl group. In an aspect, the R¹ organyl group can be a C₁ to C₂₀, a C₁ to C₁₅, a C₁ to C₁₀, or a C₁ to C₅ organyl group. In an aspect, the R¹ organyl group consisting of inert functional groups can be a C₁ to C₂₀, a C₁ to C₁₅, a C₁ to C₁₀, or a C₁ to C₅ organyl group consisting of inert functional groups. In an aspect, the R¹ hydrocarbyl group can be a C₁ to C₂₀, a C₁ to C₁₅, a C₁ to C₁₀, or a C₁ to C₅ hydrocarbyl group. In other aspects, R¹ can be an alkyl group, a substituted alkyl group, a cycloalkyl group, a substituted cycloalkyl group, an aryl group, a substituted aryl group, an aralkyl group, or a substituted aralkyl group; alternatively an alkyl group or a substituted alkyl group; alternatively, a cycloalkyl group or a substituted cycloalkyl group; alternatively, an aryl group or a substituted aryl group; alternatively, an aralkyl group or a substituted aralkyl group; alternatively, an alkyl group, a cycloalkyl group, an aryl group, or an aralkyl group; alternatively, an alkyl group; alternatively, a substituted alkyl group, alternatively, a cycloalkyl group; alternatively, a substituted cycloalkyl group; alternatively, an aryl group; alternatively, a substituted aryl group; alternatively, an aralkyl group; or alternatively, a substituted aralkyl group.

Generally, R² of the N²-phosphinyl amidines and/or the N²-phosphinyl amidine chromium complexes can be an organyl group; alternatively, an organyl group consisting of inert functional groups; or alternatively, a hydrocarbyl group. In an aspect, the R² organyl group can be a C₁ to C₂₀, a C₁ to C₁₅, a C₁ to C₁₀, or a C₁ to C₅ organyl group. In an aspect, R² organyl group consisting of inert functional groups can be a C₁ to C₂₀, a C₁ to C₁₅, a C₁ to C₁₀, or a C₁ to C₅ organyl group consisting of inert functional groups. In an aspect, R² hydrocarbyl group can be a C₁ to C₂₀, a C₁ to C₁₅, a C₁ to C₁₀, or a C₁ to C₅ hydrocarbyl group.

Generally, R³ and/or R⁴ independently can be an organyl group; alternatively, an organyl group consisting of inert functional groups; or alternatively, a hydrocarbyl group. In an aspect, the R³ and/or R⁴ organyl groups can be a C₁ to C₂₀, a C₁ to C₁₅, a C₁ to C₁₀, or a C₁ to C₅ organyl group. In an aspect, the R³ and/or R⁴ organyl groups consisting of inert functional groups can be a C₁ to C₂₀, a C₁ to C₁₅, a C₁ to C₁₀, or a C₁ to C₅ organyl group consisting of inert functional groups. In an aspect, the R³ and/or R⁴ hydrocarbyl groups can be a, a C₁ to C₂₀, a C₁ to C₁₅, a C₁ to C₁₀, or a C₁ to C₅ hydrocarbyl group. In an aspect, R³ and/or R⁴ independently can be an alkyl group, a substituted alkyl group, a cycloalkyl group, a substituted cycloalkyl group, an aryl group, a substituted aryl group, an aralkyl group, or a substituted aralkyl group; alternatively, an alkyl group or a substituted alkyl group; alternatively, a cycloalkyl group or a substituted cycloalkyl group; alternatively, an aryl group or a substituted aryl group; alternatively, an aralkyl group or a substituted aralkyl group; alternatively, an alkyl group, a cycloalkyl group, an aryl group, or an aralkyl group; alternatively, an alkyl group; alternatively, a substituted alkyl group, alternatively, a cycloalkyl group; alternatively, a substituted cycloalkyl group; alternatively, an aryl group; alternatively, a substituted aryl group; alternatively, an aralkyl group; or alternatively, a substituted aralkyl group.

Further alternatives for each of R¹, R², R³, and R⁴ can include those recited in U.S. Pat. No. 11,230,514, though have been excluded here in the interest of brevity.

As disclosed herein, it can be beneficial for the chromium complex to be soluble in the aromatic hydrocarbon solvent, in the organic reaction medium, or soluble in both the aromatic hydrocarbon solvent and the organic reaction medium. Solubility is considered at standard temperature and pressure (25° C. and 1 atm) and “soluble” means that there is no visual precipitation in a 0.001 wt. % solution. In some instances, the chromium complex is soluble in a 0.004 wt. % solution, a 0.01 wt. % solution, a 0.1 wt. % solution, or a 1 wt. % solution in the aromatic hydrocarbon solvent (or the organic reaction medium) under standard conditions. Accordingly, the chromium complex can form a solution in an aromatic hydrocarbon solvent, such as toluene, and/or can from a solution in a saturated aliphatic solvent, such as cyclohexane, and/or can form a solution in a linear α-olefin, such as 1-hexene or 1-octene.

Given a solubility of the chromium complex as discussed above, the activated catalyst composition can be relatively concentrated with respect to the chromium complex. It is contemplated that higher concentrations of chromium complex may be amenable for a storage condition of the activated catalyst, and thereby allowing a greater amount of the catalyst to be stored and transported more efficiently. In certain aspects, the concentrated activated catalyst composition can be added directly to an oligomerization process, where the scale of the oligomerization is sufficient to result in an appropriate in situ dilution with a suitable amount of the concentrated, activated catalyst composition. In certain aspects, the concentration of the chromium complex in the activated catalyst composition can be in a range from 0.001 mg/mL to 1,000 mg/mL, from 0.01 mg/mL to 100 mg/mL, or from 0.02 to 75 mg/mL. Alternatively, the concentrated activated catalyst composition may be diluted prior to the contacting step of oligomerization processes described herein.

The relative amount of the aluminoxane versus that of the chromium complex is not particularly limited. Nonetheless, molar ratios of Al:Cr in the processes for preparing activated catalyst compositions and oligomerization processes disclosed herein can range from 10:1 to 5,000:1, such as from 50:1 to 3,000:1, from 75:1 to 2,000:1, from 100:1 to 2,000:1, or from 100:1 to 1,000:1, and the like. If, for example, more than one chromium complex and/or more than one organoaluminum are employed (e.g., aluminoxane and alkylaluminum), these ratios are based on the total moles of respective chromium complexes and organoaluminums.

### Oligomerization Processes

Referring now particularly to oligomerization processes, in step (i) ethylene, an organic reaction medium, and an activated catalyst composition formed according to the processes described above can be contacted in an oligomerization reactor under oligomerization conditions. Ethylene, the organic reaction medium, and the catalyst composition (and hydrogen, if used) can be combined in any order or sequence and introduced into the oligomerization reactor separately or in any combination. This invention is not limited by the manner in which the respective feed streams are introduced into the reactor.

In an aspect, the activated catalyst composition may be stored for a period of time prior to being used e.g., in step (i) of an oligomerization process as described herein. In certain aspects, oligomerization process disclosed herein can comprise storing the activated catalyst at a storage temperature for at least 2 hours, 4 hours, 8 hours, 12 hours, 1 day, 3 days, 7 days, 14 days, or 30 days prior to its use in step (i). The storage temperature can be the same or different from the activation temperature used to prepare the activated catalyst composition. Representative and non-limiting ranges for the storage temperature of the activated catalyst composition can include from −80° C. to 20° C., from −80° C. to 10° C., from −60° C. to 0° C., from −40° C. to 0° C., or from −20° C. to 0° C.

As discussed above, the activated catalyst composition may be stored in a relatively concentrated state, which in certain aspects can be in a range from 0.1 to 10,000 ppmw, from 10 to 1,000 ppmw, from 100 to 500 ppmw, or from 200 to 300 ppmw.

Suitable values of the activation temperature and storage temperature are discussed above, without being limited thereto. However, as will be understood by those of skill in the art, the lower limit to the activation temperature for a particular process can be the freezing point of the solvents employed in the respective solutions. In this manner, the activated catalyst composition can remain a liquid while in a storage condition until use. In further aspects, the activated catalyst composition may be stored under inert atmosphere (e.g., nitrogen, argon) to preserve the catalytic properties and prevent degradation during storage.

In an aspect, after initial reactor start-up, no organic reaction medium is fed to the oligomerization reactor. Effectively, the oligomer product formed in step (ii)—discussed further below—may act as the organic reaction medium. In this aspect, the catalyst composition is highly selective for ethylene oligomerization and does not react appreciably with the hexenes, octenes, and other oligomers that are present in the oligomer product.

Any suitable organic reaction medium can be used in the disclosed oligomerization processes, including but not limited to hydrocarbons. Illustrative hydrocarbons can include, for example, saturated aliphatic hydrocarbons, aromatic hydrocarbons, and the like, as well as combinations thereof. In a particular aspect of this disclosure, the organic reaction medium can comprise (or consist essentially of, or consist of) cyclohexane. The organic reaction medium also can comprise other saturated aliphatic hydrocarbons, such as propane, butane, pentane, hexane, heptane, octane, methyl cyclohexane, or combinations thereof.

Alternatively, the organic reaction medium can be selected from the same materials as that for the aromatic hydrocarbon solvent employed in preparing the activated catalyst compositions. Thus, the organic reaction medium can comprise benzene, toluene, ethylbenzene, xylene (m-xylene, o-xylene, p-xylene), styrene, mesitylene, and the like. Combinations of two or more aromatic hydrocarbons can be utilized, if desired.

Referring now to step (ii), an oligomer product is formed in the oligomerization reactor, and the oligomer product comprises hexenes and octenes (amongst other oligomers). The oligomerization reactor in which the oligomer product is formed in step (ii) (and in which the components in step (i) are contacted) can comprise any suitable reactor. Non-limiting examples of reactor types can include a stirred tank reactor, a plug flow reactor, or any combination thereof; alternatively, a fixed bed reactor, a continuous stirred tank reactor, a loop reactor, a solution reactor, a tubular reactor, a recycle reactor, or any combination thereof. For instance, in one aspect, the oligomerization reactor can comprise a continuous stirred tank reactor, while in another aspect, the oligomerization reactor can comprise a loop reactor. In some aspects, there can be more than one reactor in series or in parallel, and including any combination of reactor types and arrangements. Moreover, the oligomerization process used to form the oligomer product can be a continuous process or a batch process, or any reactor or vessel utilized in the process can be operated continuously or batchwise.

Forming the oligomer product in the oligomerization reactor can be accomplished at any suitable oligomerization temperature and pressure. Often, the oligomer product can be formed at a minimum temperature of 0° C., 20° C., 30° C., 40° C., 45° C., or 50° C.; additionally or alternatively, at a maximum temperature of 165° C., 160° C., 150° C., 140° C., 130° C., 115° C., 100° C., or 90° C. Generally, the oligomerization temperature at which the oligomer product is formed can be in a range from any minimum temperature disclosed herein to any maximum temperature disclosed herein. Accordingly, suitable non-limiting ranges can include the following: from 0 to 165, from 20 to 160, from 20 to 115, from 40 to 160, from 40 to 140, from 40 to 115, from 50 to 150, from 50 to 140, from 50 to 130, from 50 to 100, from 60 to 115, from 70 to 100, or from 75 to 95° C. Other appropriate oligomerization temperatures and temperature ranges are readily apparent from this disclosure.

The oligomer product can be formed at a minimum pressure (or ethylene partial pressure) of 50 psig (344 kPa), 100 psig (689 kPa), 200 psig (1.4 MPa), or 250 psig (1.5 MPa); additionally, or alternatively, at a maximum pressure (or ethylene partial pressure) of 4,000 psig (27.6 MPa), 3,000 psig (20.9 MPa), 2,000 psig (13.8 MPa), or 1,500 psig (10.3 MPa). Generally, the oligomerization pressure (or ethylene partial pressure) at which the oligomer product is formed can be in a range from any minimum pressure disclosed herein to any maximum pressure disclosed herein. Accordingly, suitable non-limiting ranges can include the following: from 50 psig (344 kPa) to 4,000 psig (27.6 MPa), from 100 psig (689 kPa) to 3,000 psig (20.9 MPa), from 100 psig (689 kPa) to 2,000 psig (13.8 MPa), from 200 psig (1.4 MPa) to 2,000 psig (13.8 MPa), from 200 psig (1.4 MPa) to 1,500 psig (10.3 MPa), from 250 psig (1.5 MPa) to 1,500 psig (10.3 MPa), from 400 psig (2.6 MPa) to 1,500 psig (10.3 MPa), from 600 psig (4.1 MPa) to 1300 psig (9.0 MPa), or from 700 (4.8 MPa) to 1,200 psig (8.3 MPa). Other appropriate oligomerization pressures (or ethylene partial pressures) are readily apparent from this disclosure.

When used, hydrogen can be fed directly to the reactor, or hydrogen can be combined with an ethylene feed prior to the reactor. In the reactor, the hydrogen partial pressure can be at least 1 psig (6.9 kPa), 5 psig (34 kPa), 10 psig (69 kPa), 25 psig (172 kPa), or 50 psig (345 kPa); additionally or alternatively, a maximum hydrogen partial pressure of 2000 psig (13.8 MPa), 1750 psig (12.1 MPa), 1500 psig (10.3 MPa), 1250 psig (8.6 MPa), 1000 psig (6.9 MPa), 750 psig (5.2 MPa), 500 psig (3.4 MPa), or 400 psig (2.8 MPa). Generally, the hydrogen partial pressure can range from any minimum hydrogen partial pressure disclosed herein to any maximum hydrogen partial pressure disclosed herein. Therefore, suitable non-limiting ranges for the hydrogen partial pressure can include the following ranges: from 1 psig (6.9 kPa) to 2000 psig (13.8 MPa), from 1 psig (6.9 kPa) to 1750 psig (12.1 MPa), from 5 psig (34 kPa) to 1500 psig (10.3 MPa), from 5 psig (34 kPa) to 1250 psig (8.6 MPa), from 10 psig (69 kPa) to 1000 psig (6.9 MPa), from 10 psig (69 kPa) to 750 psig (5.2 MPa), from 10 psig (69 kPa) to 500 psig (3.5 MPa), from 25 psig (172 kPa) to 750 psig (5.2 MPa), from 25 psig (172 kPa) to 500 psig (3.4 MPa), from 25 psig (172 kPa) to 400 psig (2.8 MPa), or from 50 psig (345 kPa) to 500 psig (3.4 MPa). Other appropriate hydrogen partial pressures in the reactor for the formation of the oligomer product are readily apparent from this disclosure.

In step (iii) of the ethylene oligomerization process, an effluent stream is discharged from the oligomerization reactor, the effluent stream containing unreacted ethylene and the oligomer product. The amount of conversion of ethylene in the oligomerization reactor is not particularly limited, and generally the minimum ethylene conversion can be at least 20, 30, 35, 40, 45, or 50 wt. %, while the maximum ethylene conversion can be 99, 95, 90, 80, 75, 70, or 65 wt. %. Generally, the ethylene conversion in the reactor can range from any minimum conversion to any maximum conversion described herein. For instance, the ethylene conversion can range from 20 to 95 wt. %, from 30 to 90 wt. %, from 40 to 80 wt. %, from 50 to 70 wt. %, or from 50 to 65 wt. %. The ethylene conversion is based on the amount of ethylene entering the reactor and the amount of (unreacted) ethylene in the effluent stream.

Among other constituents, the effluent stream contains the oligomer product, which can comprise hexenes and octenes, as well as other C₄⁺ linear alpha olefins. The amount of octenes in the oligomer product typically can fall within a range from 5 to 99 wt. %, based on the total amount of oligomers in the oligomer product. In an aspect, the minimum amount of octenes in the oligomer product can be 5, 10, 20, 30 or 40 wt. %. In another aspect, the maximum amount of octenes in the oligomer product can be 99, 95, 92.5, 90, 87.5, or 85 wt. %. Generally, the amount of octenes in the oligomer product can range from any minimum amount of octenes in the oligomer product to any maximum amount of octenes in the oligomer product described herein. For instance, the amount of octenes—based on the total weight of oligomers in the oligomer product—can be in a range from 5 to 85 wt. %, from 10 to 90 wt. %, from 20 to 99 wt. %, from 30 to 95 wt. %, from 40 to 95 wt. %, from 40 to 90 wt. %, from 20 to 90 wt. %, from 30 to 87.5 wt. %, from 30 to 85 wt. %, from 40 to 87.5 wt. %, from 40 to 85 wt. %. In other aspects, the amount of octenes can be in a range from 20 to 60 wt. %, from 30 to 55 wt. %, or from 40 to 55 wt. % octenes.

Additionally, or alternatively, the oligomer product can contain any suitable amount of hexenes. In an aspect, the minimum amount of hexenes in the oligomer product can be 10, 15, 20, 25, 30, or 35 wt. %. In another aspect, the maximum amount of hexenes in the oligomer product can be 75, 65, 60, 55, or 50 wt. %. Generally, the amount of hexenes in the oligomer product can range from any minimum amount of hexenes in the oligomer product to any maximum amount of hexenes in the oligomer product described herein. For instance, the amount of hexenes—based on the total weight of oligomers in the oligomer product—can be from 10 to 75 wt. %, from 15 to 65 wt. %, from 20 to 60 wt. %, from 25 to 55 wt. %, or from 30 to 50 wt. % hexenes.

Optionally, the disclosed oligomerization processes—after step (iii)—can further comprise a step of separating (or isolating) a C₆ stream and/or a step of separating (or isolating) a C₈ stream from the oligomer product. The separated (or isolated) C₆ stream can contain any suitable amount of hexene(s), such as at least 96, at least 97, at least 98, or at least 99 wt. % hexene(s), based on the total weight of the C₆ stream, and the separated (or isolated) C₈ stream can contain any suitable amount of octene(s), such as at least 96, at least 97, at least 98, or at least 99 wt. % octene(s), based on the total weight of the C₈ stream.

Generally, a vast majority of these C₆ and C₈ streams are the desirable α-olefin products, 1-hexene and 1-octene, respectively. While not limited thereto, the C₆ stream can contain, for example, at least 90 wt. % 1-hexene, and more often at least 92.5 wt. %, at least 95 wt. %, at least 97.5 wt. %, at least 98 wt. %, at least 98.5 wt. %, or at least 99 wt. % 1-hexene, based on the total weight of the hexene(s) in the C₆ stream. Likewise, the C₈ stream can contain, for example, at least 95 wt. % 1-octene, and more often, at least 96 wt. %, at least 96.5 wt. %, at least 97 wt. %, at least 97.5 wt. %, at least 98 wt. %, or 98.5 wt. % 1-octene, based on the total weight of the octene(s) in the C₈ stream.

As evidenced by the Examples below, the performance of oligomerization processes disclosed herein can be improved with the use of the activated catalyst compositions prepared by processes described above. In certain aspects, the oligomerization reaction can run cleaner, with the production of fewer fouling solids during the oligomerization. Fouling solids may build up along the interior of the oligomerization reactor and eventually require shutdown and cleaning of equipment, in addition to contaminating the oligomer product. Oligomerization processes disclosed herein can generate an amount of fouling solids less than (at least 10% less than, at least 20% less than, at least 30% less than, at least 40% less than, at least 50% less than, at least 60% less than, at least 70% less than, at least 80% less than, or at least 90% less than) that of an otherwise identical oligomerization process in which the activation temperature and/or the storage temperature is greater than or equal to 20° C. In this comparison, the catalyst composition and oligomerization conditions are the same; the only difference is the activation temperature of the catalyst composition, or the storage temperature of the catalyst composition, or both.

It is further surprising that the reduction of fouling solids as described above is accomplished without a detrimental impact on the productivity, selectivity, yield, or activity of the activated catalyst compositions in oligomerization processes. Additionally, or alternatively, aspects of oligomerization processes disclosed herein can demonstrate a productivity that is greater than (e.g., from 5% to 50% greater than) that of, or within 20% of (e.g., within 10% of or within 5% of) an otherwise identical oligomerization process in which the activation temperature and/or the storage temperature is greater than or equal to 20° C. As above, in this comparison, the catalyst composition and oligomerization conditions are the same; the only difference is the activation temperature of the catalyst composition, or the storage temperature of the catalyst composition, or both.

Additionally or alternatively, the activity of the activated catalyst composition can be greater than (e.g., from 5% to 50% greater than) that of, or within 20% of (e.g., within 10% of or within 5% of) an otherwise identical catalyst system in which the activation temperature and/or the storage temperature is greater than or equal to 20° C. As above, in this comparison, the catalyst composition and oligomerization conditions used to determine activity are the same; the only difference is the activation temperature of the catalyst composition, or the storage temperature of the catalyst composition, or both.

### EXAMPLES

The invention is further illustrated by the following examples, which are not to be construed in any way as imposing limitations to the scope of this invention. Various other aspects, modifications, and equivalents thereof which, after reading the description herein, can suggest themselves to one of ordinary skill in the art without departing from the spirit of the present invention or the scope of the appended claims.

### Examples 1-13

For each of Examples 1-6, a slurry of a representative chromium complex (a N²-phosphinyl guanidine chromium(III) trichloride tetrahydrofuran complex) was prepared in m-xylene and the temperature adjusted to the activation temperature as noted in Table 1. Triethylaluminum (0.8 mL) was then added to the slurry with stirring to form a homogenous blue pre-catalyst solution. After 15 minutes, MMAO (7 mL) in solution with cyclohexane was added to the pre-catalyst solution with varying conditions (time and temperature) and stirred for 45 minutes to form the activated catalyst composition. The activated catalyst composition was then immediately fed to a continuous stirred tank reactor and pressurized with ethylene. Each of the oligomerization reactions were conducted at a temperature of 85° C. and a pressure of 875 psig. Ethylene was fed to the reactor on demand, and the reactions were terminated by degassing after 15 min. Products were analyzed by gas chromatography, using an internal standard.

Results from the ethylene oligomerization experiments of Examples 1-6 are summarized in Table 1. Comparing Example 1 (catalyst activated at 20° C.) and Example 6 (catalyst activated at −20° C.), it was observed that, surprisingly, the amount of fouling solids present in the product stream decreased by a factor of nearly 10, from 0.17 to 0.02. Moreover, Example 6 also demonstrated an increased total productivity and ethylene conversion, while maintaining similar product ratios and purity for 1-hexene and 1-octene products. Examples 3-4 (catalyst activated at 0° C.) demonstrated a sharply increased ethylene conversion and total productivity, while also improving the relative amount of C₆ and C₈ produced compared to higher molecular weight products.

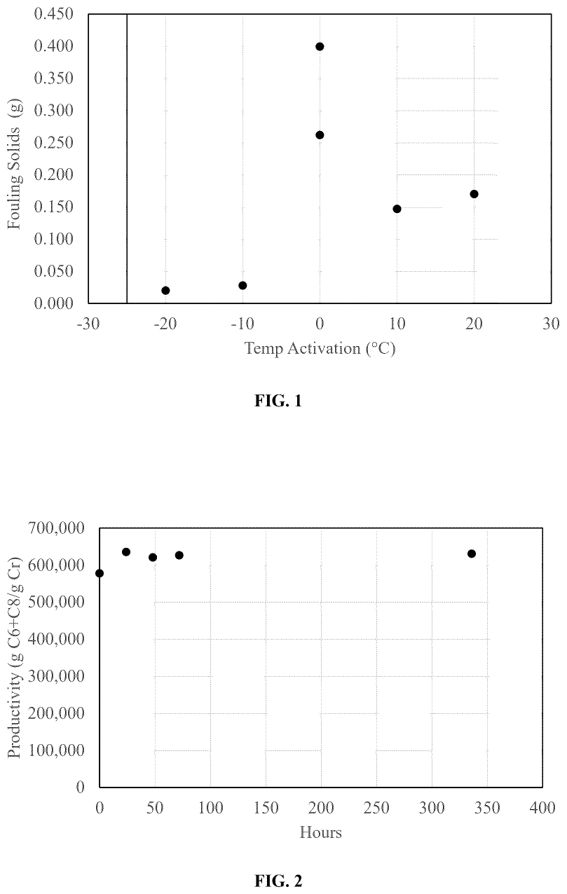

FIG. 1 illustrates the amount of fouling solids present in the reactor following each of Examples 1-6, according to the temperature at which the catalyst composition was activated. Examples 5-6 unexpectedly showed a sharply reduced amount of fouling solids which can be attributed to the low temperature activation of the catalyst composition. As above, it is observed from the values in Table 1 that this reduced amount of fouling solids is not the product of a less active catalyst, as the productivity, catalytic activity, yield, and selectivity for each of these Examples remains within the same range as or in most instances superior to that of Example 1.

TABLE 1
Reaction conditions and results for Examples 1-6.

| Example | 1 | 2 | 3 | 4 | 5 | 6 |
|---|---|---|---|---|---|---|
| Reaction Conditions | | | | | | |
| Catalyst Activation | 20 | 10 | 0 | 0 | −10 | −20 |
| Temperature (° C.) | | | | | | |
| Al:Cr | 303 | 307 | 293 | 311 | 303 | 303 |
| Residence Time (min) | 270 | 210 | 60 | 240 | 240 | 120 |
| Total Al:Cr | 423 | 428 | 408 | 433 | 423 | 423 |
| Product stream | | | | | | |
| Diluent (g/h) | 300 | 306 | 290 | 328 | 310 | 318 |
| Ethylene (g/h) | 255 | 248 | 240 | 246 | 250 | 253 |
| Cr (ppmw) | 0.35 | 0.34 | 0.37 | 0.33 | 0.34 | 0.33 |
| Al (ppmw) | 54 | 55 | 57 | 53 | 54 | 53 |
| H₂ (g/h) | 0.108 | 0.108 | 0.108 | 0.108 | 0.108 | 0.108 |
| H₂:Cr (mol/mol) | 14,545 | 14,717 | 14,051 | 14,894 | 14,545 | 14,545 |
| Product Characterization | | | | | | |
| Total Productivity | 614,680 | 627,355 | 623,565 | 644,287 | 627,659 | 622,484 |
| (g NAO/g Cr) | | | | | | |
| 1-hexene | 258,715 | 265,176 | 253,050 | 284,058 | 268,004 | 260,708 |
| (g NAO/g Cr) | | | | | | |
| 1-octene | 355,965 | 362,179 | 370,515 | 360,229 | 359,655 | 361,776 |
| (g NAO/g Cr) | | | | | | |
| Ethylene Conv. (%) | 54.64% | 57.02% | 61.07% | 57.98% | 57.61% | 56.33% |
| Selectivity (%) | 89.53% | 89.55% | 87.73% | 89.92% | 89.96% | 89.06% |
| C₆ Purity (%) | 88.97% | 89.43% | 89.07% | 89.83% | 89.03% | 88.94% |
| C₈ Purity (%) | 96.50% | 97.28% | 97.27% | 97.32% | 96.58% | 96.69% |
| C₆ Yield (wt. %) | 40.34% | 40.36% | 39.00% | 42.07% | 40.87% | 40.19% |
| C₈ Yield (wt. %) | 51.22% | 50.73% | 52.23% | 49.22% | 50.56% | 51.31% |
| C₁₀₊ Yield (wt. %) | 8.44% | 8.91% | 8.77% | 8.71% | 8.57% | 8.50% |
| Fouling Solids (wt. %) | 0.02% | 0.02% | 0.05% | 0.03% | 0.00% | 0.00% |
| Fouling Solids (g) | 0.170 | 0.147 | 0.399 | 0.262 | 0.028 | 0.020 |
| Reaction Efficiency | 1.96 | 1.98 | 2.06 | 2.01 | 2.00 | 1.98 |
| (lb/g/h) | | | | | | |
| C₈/C₆ Ratio | 1.27 | 1.26 | 1.34 | 1.17 | 1.24 | 1.28 |

Following the surprising results from Examples 1-6 as described above, and Example 6 particularly (catalyst activation at −20° C.), Examples 7-11 were conducted to evaluate the storage stability of activated catalyst prepared in a similar manner. Examples 7-11 were performed at the same oligomerization conditions as Examples 1-6. Catalyst compositions of Examples 7-11 were activated with slight variations to the activation conditions. The first activation step combining the chromium catalyst with triethylaluminum (2.8 mL) to produce a homogeneous blue solution was conducted for a period of only 5 minutes, as compared to 15 minutes for Examples 1-6. The pre-catalyst solution of Example 7 was prepared at 20° C., whereas those of Examples 8-11 were prepared at −20° C. As above for Examples 1-6, an amount of MMAO (7 mL) was then added to form the activated catalyst composition maintaining the relevant temperature.

The activated catalyst composition of Example 7 was prepared at ambient temperature (20° C.) and used within the oligomerization process at 15 minutes. Examples 8-11 prepared activated catalyst compositions at −20° C. as noted above, however, also delayed their use in oligomerization processes well beyond the initial activation period of 15 to 45 minutes seen in Examples 1-7. Examples 8-11 stored the activated catalyst composition for 1 day, 2 days, 3 days, and 14 days, respectively, prior to feeding the catalyst into an oligomerization reactor.

Surprisingly, as drawn from the data in Table 2 below and illustrated in FIG. 2, the activated catalysts maintained or improved their effectiveness (as measured by total productivity, ethylene conversion, selectivity to C₆/C₈ products, and the amount of fouling solids present in the product stream). This result was particularly surprising in view of conventional catalyst activation processes conducted at or above room temperature where the activated catalyst begins to lose effectiveness within several hours, to the point that fresh catalyst conventionally had been prepared immediately before each oligomerization run.

TABLE 2
Reaction conditions and results for Examples 7-11.

| Example | 7 | 8 | 9 | 10 | 11 |
|---|---|---|---|---|---|
| Reaction Conditions | | | | | |
| Activated Catalyst | 15 min | 1 day | 2 days | 3 days | 14 days |
| Storage Time | | | | | |
| Storage Temperature (° C.) | 20 | −20 | −20 | −20 | −20 |
| Al:Cr | 311 | 311 | 311 | 311 | 311 |
| Line Out Average Time | 360 | 300 | 450 | 450 | 390 |
| Total Al:Cr | 738 | 738 | 738 | 738 | 738 |
| Diluent (g/h) | 319 | 327 | 311 | 325 | 320 |
| Ethylene (g/h) | 251 | 247 | 247 | 249 | 251 |
| Cr (ppmw) | 0.318 | 0.317 | 0.325 | 0.316 | 0.318 |
| Al (ppmw) | 51 | 51 | 52 | 51 | 51 |
| H₂ (g/h) | 0.108 | 0.108 | 0.108 | 0.108 | 0.108 |
| H₂:Cr (mol/mol) | 14,955 | 14,955 | 14,955 | 14,955 | 14,955 |
| Mean residence time (min) | 36 | 36 | 37 | 36 | 36 |
| Product Characterization | | | | | |
| Total Productivity | 577,908 | 635,438 | 620,911 | 626,413 | 630,644 |
| (g NAO/g Cr) | | | | | |
| 1-hexene | 225,334 | 270,348 | 266,780 | 269,656 | 265,706 |
| (g NAO/g Cr) | | | | | |
| 1-octene | 352,574 | 365,091 | 354,131 | 356,757 | 364,938 |
| (g NAO/g Cr) | | | | | |
| Ethylene Conv. (%) | 50.8% | 57.2% | 55.7% | 55.5% | 55.4% |
| C₆ Purity (%) | 89.1% | 89.7% | 89.8% | 89.8% | 89.6% |
| C₈ Purity (%) | 97.3% | 97.3% | 97.3% | 97.3% | 97.2% |
| C₆ Yield (wt. %) | 37.2% | 40.2% | 40.5% | 40.6% | 40.0% |
| C₈ Yield (wt. %) | 53.4% | 50.2% | 49.7% | 49.8% | 50.7% |
| C₁₀₊ Yield (wt. %) | 9.4% | 9.6% | 9.8% | 9.6% | 9.3% |
| Fouling Solids (wt. %) | 0.0% | 0.0% | 0.0% | 0.0% | 0.0% |
| Fouling Solids (g) | 0.162 | 0.028 | 0.029 | 0.028 | 0.065 |
| Rx Eff (lb/g/h) | 1.8 | 2.0 | 1.9 | 1.9 | 2.0 |
| C₈/C₆ Ratio | 1.6 | 1.4 | 1.3 | 1.3 | 1.4 |

Examples 12-13 represent ethylene oligomerization reactions performed in duplicate under batch conditions using a N²-phosphinyl guanidine chromium(III) trichloride tetrahydrofuran complex as in Examples 1-11.

A stock solution of the catalyst complex (0.0037 g), nonane (1 g), and meta-xylene (1.0034 g) were stirred until the catalyst complex was fully dissolved. MMAO (1.2130 g) was then added with stirring for 24 h to activate the catalyst solution. Afterwards, 200 mL of cyclohexane was added to a charger along with the contents of the stock solution. Once the reactor was pumped down, the charger was attached to the top of the reactor, and the contents were emptied into the reactor with static vacuum. Once the reactor reached a set temperature of 85° C., the vessel was filled with 50 psi hydrogen gas, and 800 psi of ethylene was then added. The stirrer was turned on to begin the reaction, which ran for 30 minutes. Once completed, the reactor was cooled, vented, and a sample was collected and analyzed by GC. Solid materials were also collected and weighed.

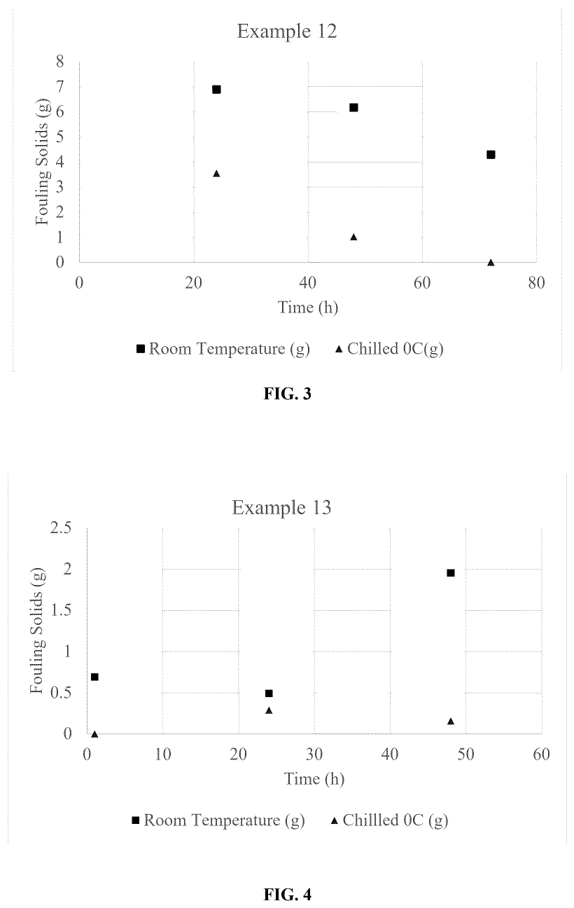

The amount of fouling solids within each of the crude products was analyzed by gas chromatography at 3 different time points as shown in FIGS. 3-4, using an internal standard. As shown, Examples 12 and 13 both confirm the general trend shown in Examples 1-11 above, demonstrating that an activation temperature less than 20° C. (here 0° C.) again resulted in a reduced amount of fouling solids in the crude product.

### ASPECTS AND EMBODIMENTS

The invention is described herein with reference to numerous aspects and specific examples. Many variations will suggest themselves to those skilled in the art in light of the detailed description. All such obvious variations are within the full intended scope of the appended claims. Other aspects of the invention can include, but are not limited to, the following (aspects are described as “comprising” but, alternatively, can “consist essentially of” or “consist of”):

Aspect 1. A process for preparing an activated catalyst composition, the process comprising (a) contacting a mixture of a chromium complex in an aromatic hydrocarbon solvent with an aluminoxane at an activation temperature to form the activated catalyst composition; and (b) optionally, reducing a temperature of the activated catalyst composition to a storage temperature; wherein at least one of the activation temperature and the storage temperature is less than 20° C. (e.g., less than 0° C.; or in a range from −80° C. to 20° C., or in a range from −40° C. to 0° C.); and the activated catalyst composition is stable at the storage temperature for greater than 24 hours (e.g., from 2 to 30 days) without loss of activity.

Aspect 2. The process defined in aspect 1, wherein the activation temperature is less than 20° C. (e.g., less than 0° C.; or in a range from −80° C. to 20° C., or in a range from −40° C. to 0° C.).

Aspect 3. The process defined in aspect 1, wherein the activation temperature is greater than 20° C., and the process comprises reducing the temperature of the activated catalyst composition to the storage temperature of less than 20° C.

Aspect 4. The process defined in any one of aspects 1-3, wherein the aluminoxane comprises methylaluminoxane (MAO), ethylaluminoxane, modified methylaluminoxane (MMAO), n-propylaluminoxane, iso-propyl-aluminoxane, n-butylaluminoxane, sec-butylaluminoxane, iso-butylaluminoxane, t-butylaluminoxane, 1-pentylaluminoxane, 2-entylaluminoxane, 3-pentyl-aluminoxane, iso-pentyl-aluminoxane, neopentylaluminoxane, or any combination thereof.

Aspect 5. The process defined in any one of aspects 1-4, wherein the mixture is contacted with a solution of the aluminoxane in a saturated aliphatic hydrocarbon.

Aspect 6. The process defined in any one of aspects 1-5, wherein the mixture further comprises an alkylaluminum compound.

Aspect 7. The process defined in aspect 6, wherein the alkylaluminum compound comprises trimethylaluminum (TMA), triethylaluminum (TEA), tripropylaluminum, tri-n-butylaluminum, triisobutylaluminum (TIBA), trihexylaluminum, trioctylaluminum, or any combination thereof.

Aspect 8. The process defined in any one of aspects 1-7, wherein an Al to Cr molar ratio of the activated catalyst composition is in a range from 10:1 to 5,000:1, from 50:1 to 3,000:1, from 75:1 to 3,000:1, from 75:1 to 2,000:1, from 100:1 to 2,000:1, or from 100:1 to 1,000:1.

Aspect 9. The process defined in any one of aspects 1-8, wherein the aromatic hydrocarbon solvent comprises benzene, toluene, xylenes, cumene, ethylbenzenes, or combinations thereof.

Aspect 10. The process defined in any one of aspects 1-8, wherein the aromatic hydrocarbon solvent comprises o-xylene, m-xylene, p-xylene, or a combination thereof.

Aspect 11. The process defined in any one of aspects 1-10, wherein a concentration of the chromium complex in the catalyst composition is in a range from 0.01 to 100 mg/mL, or from 0.02 to 75 mg/mL.

Aspect 12. The process defined in any one of aspects 1-11, wherein the chromium complex has the following formula:

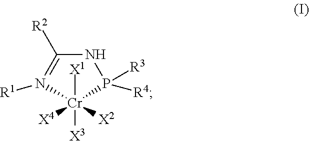

wherein:

X¹ to X⁴ independently are a monoanionic ligand; and R¹ to R⁴ independently are a substituted or unsubstituted C₁-C₁₈ hydrocarbyl group.

Aspect 13. The process defined in any one of aspects 1-12, wherein the chromium complex comprises a PNP ligand, an SNS ligand, or a pyrrole ligand.

Aspect 14. The process defined in any one of aspects 1-13, wherein the chromium complex comprises an N²-phosphinyl guanidine chromium complex, an N²-phosphinyl formamidine chromium complex, an N²-phosphinyl amidine chromium complex, or any combination thereof.

Aspect 15. An oligomerization process comprising (i) contacting ethylene, the activated catalyst composition defined in any one of aspects 1-14, an organic reaction medium, and optionally hydrogen, in an oligomerization reactor under oligomerization conditions; (ii) forming an oligomer product in the oligomerization reactor; and (iii) discharging an effluent stream from the oligomerization reactor, the effluent stream comprising unreacted ethylene and the oligomer product.

Aspect 16. The oligomerization process defined in aspect 15, further comprising storing the activated catalyst composition at the storage temperature for at least 48 hours (e.g., at least 7 days, at least 14 days, at least 30 days) prior to the contacting step (i).

Aspect 17. The oligomerization process defined in aspect 16, wherein the storage temperature is less than or equal to the activation temperature (less than 0° C., or in a range from −80° C. to 20° C., or in a range from −40° C. to 0° C.).

Aspect 18. The oligomerization process defined in aspect 16 or 17, wherein the activated catalyst composition is stored in an inert atmosphere.

Aspect 19. The oligomerization process defined in any one of aspects 15-18, wherein storage conditions comprise storing the activated catalyst composition at a concentration in a range from 1 to 10,000 ppmw, from 10 to 5,000 ppmw, or from 200 to 500 ppmw (ppm by weight).

Aspect 20. The oligomerization process defined in any one of aspects 15-19, further comprising diluting the activated catalyst composition prior to the contacting step (i).

Aspect 21. The oligomerization process defined in any one of aspects 15-20, wherein an amount of fouling solids produced during the oligomerization process is less than (e.g., at least 10% less than, at least 30% less than, at least 50% less than, at least 90% less than) that of an otherwise identical oligomerization process in which the activation temperature and/or the storage temperature is greater than or equal to 20° C.

Aspect 22. The oligomerization process defined in any one of aspects 15-21, wherein an activity of the activated catalyst composition is greater than that (or within 10%) of an otherwise identical catalyst system in which the activation temperature and/or the storage temperature is greater than or equal to 20° C.

Aspect 23. The oligomerization process defined in any one of aspects 17-22, wherein a productivity of the oligomerization process is greater than that (or within 10%) of an otherwise identical oligomerization process in which the activation temperature and/or the storage temperature is greater than or equal to 20° C.

Aspect 24. The oligomerization process defined in any one of aspects 15-23 wherein the oligomerization reactor is a stirred tank reactor, a plug flow reactor, a fixed bed reactor, a continuous stirred tank reactor, a loop reactor, a solution reactor, a tubular reactor, a recycle reactor, or any combination thereof.

Aspect 25. The oligomerization process defined in any one of aspects 15-24, wherein the oligomerization reactor comprises more than one reactor in series or in parallel.

Aspect 26. The oligomerization process defined in any one of aspects 15-25, wherein the oligomer product is formed at any suitable reaction temperature, e.g., from 40° C. to 115° C., from 60° C. to 115° C., from 70° C. to 100° C., or from 75° C. to 95° C.

Aspect 27. The oligomerization process defined in any one of aspects 15-26, wherein the oligomer product is formed at any suitable reaction pressure, e.g., from 400 psig to 1500 psig, from 600 psig to 1300 psig, or from 700 to 1200 psig.

Aspect 28. The oligomerization process defined in any one of aspects 15-27, wherein hydrogen is contacted in the oligomerization reactor.

Aspect 29. The oligomerization process defined in any one of aspects 15-28, wherein hydrogen and ethylene are combined and introduced into the reactor separately from the activated catalyst composition.

Aspect 30. The oligomerization process defined in any one of aspects 15-29, wherein the organic reaction medium comprises a saturated aliphatic hydrocarbon, an aromatic hydrocarbon, or any combination thereof.

Aspect 31. The oligomerization process defined in any one of aspects 15-30, wherein the organic reaction medium comprises a saturated aliphatic hydrocarbon, e.g., propane, butane, pentane, hexane, heptane, octane, cyclohexane, methyl cyclohexane, or combinations thereof.

Aspect 32. The oligomerization process defined in any one of aspects 15-31, wherein the organic reaction medium comprises cyclohexane.

Aspect 33. The oligomerization process defined in any one of aspects 15-32, wherein the organic reaction medium comprises an aromatic hydrocarbon, e.g., benzene, toluene, xylene, cumene, ethylbenzene, or combinations thereof.

Aspect 34. The oligomerization process defined in any one of aspects 15-33, wherein the oligomerization reactor has any ethylene conversion disclosed herein, e.g., at least 20, 30, 35, 40, 45, or 50 wt. %; a maximum of 99, 95, 90, 80, 75, 70, or 65 wt. %; or from 20 to 95 wt. %, from 30 to 90 wt. %, from 40 to 80 wt. %, from 50 to 70 wt. %, or from 50 to 65 wt. % conversion, based on the amount of ethylene entering the reactor and the amount of ethylene in the effluent stream.

Aspect 35. The oligomerization process defined in any one of aspects 15-34, wherein the oligomer product comprises hexenes.

Aspect 36. The oligomerization process defined in aspect 35, wherein the oligomer product comprises any amount of hexenes disclosed herein, e.g., at least 5, 10, 20, 30 or 40 wt. %; a maximum of 99, 95, 92.5, 90, 87.5, or 85 wt. %; or from 5 to 85 wt. %, from 10 to 90 wt. %, from 20 to 99 wt. %, from 30 to 95 wt. %, from 40 to 95 wt. %, from 40 to 90 wt. %, from 20 to 90 wt. %, from 30 to 87.5 wt. %, from 30 to 85 wt. %, from 40 to 87.5 wt. %, from 40 to 85 wt. %, from 20 to 60 wt. %, from 30 to 55 wt. %, or from 40 to 55 wt. % hexenes, based on the total amount of oligomers in the oligomer product.

Aspect 37. The oligomerization process defined in any one of aspects 1-36, wherein the oligomer product comprises octenes.

Aspect 38. The oligomerization process defined in aspect 37, wherein the oligomer product comprises any amount of octenes disclosed herein, e.g., at least 5, 10, 20, 30 or 40 wt. %; a maximum of 99, 95, 92.5, 90, 87.5, or 85 wt. %; or from 5 to 85 wt. %, from 10 to 90 wt. %, from 20 to 99 wt. %, from 30 to 95 wt. %, from 40 to 95 wt. %, from 40 to 90 wt. %, from 20 to 90 wt. %, from 30 to 87.5 wt. %, from 30 to 85 wt. %, from 40 to 87.5 wt. %, from 40 to 85 wt. %, from 20 to 60 wt. %, from 30 to 55 wt. %, or from 40 to 55 wt. % octenes, based on the total amount of oligomers in the oligomer product.

What is claimed is:

1. A process for preparing an activated catalyst composition, the process comprising:
(a) contacting a mixture of a chromium complex in an aromatic hydrocarbon solvent with an aluminoxane at an activation temperature to form the activated catalyst composition; and
(b) optionally, reducing a temperature of the activated catalyst composition to a storage temperature; wherein:
at least one of the activation temperature and the storage temperature is less than 20° C.; and
the activated catalyst composition is stable at the storage temperature for greater than 24 hours.

2. The process of claim 1, wherein the activation temperature is less than 20° C.

3. The process of claim 1, wherein the activation temperature is greater than 20° C., and the process further comprises reducing the temperature of the activated catalyst composition to the storage temperature of less than 20° C.

4. The process of claim 1, wherein the aluminoxane comprises methylaluminoxane (MAO), ethylaluminoxane, modified methylaluminoxane (MMAO), n-propylaluminoxane, iso-propyl-aluminoxane, n-butylaluminoxane, sec-butylaluminoxane, iso-butylaluminoxane, t-butylaluminoxane, 1-pentylaluminoxane, 2-entylaluminoxane, 3-pentyl-aluminoxane, iso-pentyl-aluminoxane, neopentylaluminoxane, or any combination thereof.

5. The process of claim 1, wherein the mixture further comprises an alkylaluminum compound selected from trimethylaluminum (TMA), triethylaluminum (TEA), tripropylaluminum, tri-n-butylaluminum, triisobutylaluminum (TIBA), trihexylaluminum, trioctylaluminum, and any combination thereof.

6. The process of claim 1, wherein an Al to Cr molar ratio of the activated catalyst composition is in a range from 10:1 to 5,000:1.

7. The process of claim 1, wherein the aromatic hydrocarbon solvent comprises m-xylene, o-xylene, p-xylene, or a combination thereof.

8. The process of claim 1, wherein the chromium complex has the following formula: 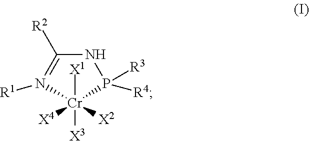
wherein:
X¹ to X⁴ independently are a monoanionic ligand; and
R¹ to R⁴ independently are a substituted or unsubstituted C₁-C₁₈ hydrocarbyl group.

9. The process of claim 1, wherein the chromium complex comprises a PNP ligand, an SNS ligand, or a pyrrole ligand.

10. The process of claim 1, wherein the chromium complex comprises an N²-phosphinyl guanidine chromium complex, an N²-phosphinyl formamidine chromium complex, an N²-phosphinyl amidine chromium complex, or any combination thereof.

11. An oligomerization process comprising:
(I) performing the process for preparing the activated catalyst composition of claim 1;
(II) contacting ethylene, the activated catalyst composition, an organic reaction medium, and optionally hydrogen, in an oligomerization reactor under oligomerization conditions;
(III) forming an oligomer product in the oligomerization reactor; and
(IV) discharging an effluent stream from the oligomerization reactor, the effluent stream comprising unreacted ethylene and the oligomer product.

12. The oligomerization process of claim 11, further comprising storing the activated catalyst composition at the storage temperature for at least 48 hours prior to the contacting step (II).

13. The oligomerization process of claim 12, wherein the storage temperature is less than or equal to the activation temperature.

14. The oligomerization process of claim 11, further comprising diluting the activated catalyst composition prior to the contacting step (II).

15. The oligomerization process of claim 11, wherein an amount of fouling solids produced during the oligomerization process is at least 10% less than that of an otherwise identical oligomerization process in which the activation temperature and/or the storage temperature is greater than or equal to 20° C.

16. The oligomerization process of claim 11, wherein an activity of the activated catalyst composition is greater than that of the otherwise identical catalyst system in which the activation temperature and/or the storage temperature is greater than or equal to 20° C.

17. The oligomerization process of claim 11, wherein a productivity of the oligomerization process is greater than that of the otherwise identical oligomerization process in which the activation temperature and/or the storage temperature is greater than or equal to 20° C.

18. The oligomerization process of claim 11, wherein the organic reaction medium comprises cyclohexane.

19. The oligomerization process of claim 11, wherein the oligomer product comprises from 30 to 55 wt. % hexenes.

20. The oligomerization process of claim 11, wherein the oligomer product comprises from 40 to 55 wt. % octenes.
